(19) United States
(12) Patent Application Publication
(10) Pub. No.: US 2026/0033783 A1
(43) Pub. Date: Feb. 5, 2026

(54) DETECTION OF SIGNAL VARIATIONS DUE TO PRESSURE ON ANALYTE SENSOR

(71) Applicant: Laxmi Therapeutic Devices, Inc., Goleta, CA (US)

(72) Inventors: Thomas Metzmaker, Goleta, CA (US); William Peter Van Antwerp, Santa Clarita, CA (US); Matthew Yavorsky, Granada Hills, CA (US); Peter Rule, Los Altos Hills, CA (US)

(21) Appl. No.: 19/288,372

(22) Filed: Aug. 1, 2025

Related U.S. Application Data
(60) Provisional application No. 63/678,913, filed on Aug. 2, 2024.

(51) Int. Cl. A61B 5/00; A61B 5/145

(52) U.S. Cl. CPC A61B 5/6843; A61B 5/1451; A61B 5/14532; A61B 5/6833; A61B 5/746

(57) ABSTRACT

A monitor for determining analyte concentrations in vivo includes a housing configured to adhere to a patient's skin, a sensor member configured to extend from the housing into the patient's skin, and a pressure sensor positionable between part of the housing and part of the patient's skin. When pressure is exerted on the housing towards the patient's skin, the pressure sensor is configured to measure a magnitude of the pressure exerted on the housing.

### CROSS-REFERENCE TO RELATED APPLICATION(S)

The present application claims priority to and the benefit of both U.S. Provisional Patent Application No. 63/678,913, filed Aug. 2, 2024, the contents of which are hereby incorporated by reference in their entirety.

### BACKGROUND

### Field

The present disclosure relates to medical devices such as analyte sensors, which can be used to measure analytes, such as glucose levels, in the body of a subject. More specifically, the present disclosure is directed to improvements in such analyte sensors, and in particular, systems and methods for helping to determine whether signal artifacts or other signal variations are due to or induced by compression or other pressures applied onto the analyte sensor or onto the tissue under the analyte sensor.

### Description of Related Art

Monitoring different analytes in the human body can be used for various diagnostic reasons. In particular, monitoring glucose levels is important for individuals suffering from type 1 or type 2 diabetes. People with type 1 diabetes are unable to produce insulin or produce very little insulin, while people with type 2 diabetes are resistant to the effects of insulin. Insulin is a hormone produced by the pancreas that helps regulate the flow of blood glucose from the bloodstream into the cells in the body where it can be used as a fuel. Without insulin, blood glucose can build up in the blood and lead to various symptoms and complications, including fatigue, frequent infections, cardiovascular disease, nerve damage, kidney damage, eye damage, and other issues. Individuals with type 1 or type 2 diabetes need to monitor their glucose levels in order to avoid these symptoms and complications.

Analyte monitors, and in particular, glucose monitors for the monitoring of glucose levels for the management of diabetes, are constantly being developed and improved. Although there are several platforms for monitoring analytes such as glucose available on the market, there is still a need to improve their precision and wearability. Additionally, there is a desire to provide more robust, less error-prone, and/or generally more effective continuous glucose monitors that may be attached to a patient's body for a prolonged period of time, as well as glucose monitor features that can be used together with such improved monitor and monitor designs.

### SUMMARY

Many continuous glucose monitors are intended to be worn on a patient's skin for a duration of multiple days or weeks. Most or all commercially available glucose sensors on the market today sense glucose in interstitial fluid (ISF) below the surface of the skin. Such sensing or monitoring therefore typically involves an initial step of inserting a sensing portion of a sensor member of the glucose monitor under the patient's skin. For the most part, this insertion step will involve puncturing the surface of the skin, for example, with a separate needle, for example, on an applicator to provide access for inserting the sensor. Thereafter, the needle or other sharp may be retracted, while the sensor member stays in place under the patient's skin. The rest of the monitor may be adhered to the patient's skin above the sensor member, and may be physically connected to the sensor member, in order to hold the sensor member in place under the skin, to protect the sensor member from environmental and other external conditions, and to provide and house electronics associated with the analyte monitoring, for example, a battery to provide power to the system, a processor and other circuitry to record and/or analyze electrical signals received from the sensor member, and/or a transmitter to transmit data to an offsite location, for example, a cellular phone or server configured to receive and further process the collected data.

As noted above, sensor members for continuous glucose monitors are positioned under the surface of a patient's skin, and monitor glucose levels in the area surrounding the sensor member. Some of these sensor members, particularly those associated with embodiments of the invention, may depend on glucose diffusion in the interstitial fluid around the site of where sensing regions of the sensor member are located to be representative of the body's glucose. Such glucose level representations determined through the sensor readings are generally accurate.

However, in some instances, the local tissue surrounding the sensor member may be overly compressed, which causes there to be less interstitial fluid and less diffusion in the compressed region, which may make the local glucose levels of the tissue surrounding the sensor member to be significantly lower than in other parts of the body. Compression or other pressure may be caused, for example, by leaning the analyte sensor against another object, laying on the sensor site when the patient is asleep, or any of various other reasons.

When leveraging or relying on only the general sensor signal, such deviations in sensor signals caused by compression or other pressure at the sensor site may not be easily identifiable. Particularly, a main function of glucose sensors is to identify hypoglycemia, or drops in blood sugar or blood glucose levels, so that appropriate measures, such as providing insulin to the patient, can be taken. It may be difficult to distinguish between a lowered signal that is caused by compression at the sensor site, as opposed to a lowered signal that is caused by hypoglycemia. Indeed, existing systems often treat low signals due to compression as a real low signal caused by low blood glucose, since they are often indistinguishable, which may for example, trigger an alarm. This commonly results in patients or other users receiving low glucose alarms at night, waking them from their sleep with presumed hypoglycemia, which may be an annoyance to the patients since they are not actually in danger due to the low signals not actually being caused by low blood glucose levels. Therefore, it is important to be able to distinguish between these two types of low signals, so that proper care to the patient is not interrupted.

According to embodiments of the invention, “compression low” signals, or signals that are low or otherwise varied due to compression or other pressure at the sensor site, can be more effectively detected or identified and distinguished from regular monitoring signals, so that treatment is not interrupted or improperly administered due to such compression low signals. For example, in some embodiments, one or more additional sensors can be incorporated into, or other types of measurements can be taken by, the continuous glucose monitor or other wearable device to identify situations where compression is increased, so that treatment of the patient can be adjusted accordingly.

According to an embodiment of the invention, a monitor for determining analyte concentrations in vivo includes a housing configured to adhere to a patient's skin, a sensor member configured to extend from the housing into the patient's skin, and a pressure sensor positionable between part of the housing and part of the patient's skin. When pressure is exerted on the housing towards the patient's skin, the pressure sensor is configured to measure a magnitude of the pressure exerted on the housing.

The pressure sensor may be formed on a surface of the housing configured to face towards the patient's skin. The pressure sensor may be integrally formed on the surface of the housing configured to face towards the patient's skin.

The monitor may further include an adhesive layer configured to be positioned between the housing and the patient's skin, to facilitate adhesion of the monitor to the patient's skin. An opening may be formed in the adhesive layer, and the pressure sensor may extend from the housing through the opening, such that the pressure sensor is configured to directly contact the patient's skin. The adhesive layer may have an outer surface that faces away from the housing and that is configured to engage the patient's skin, and at least part of the pressure sensor may be configured to extend axially past the outer surface of the adhesive layer, such that when the adhesive layer contacts the patient's skin, the pressure sensor is at least partially actuated or otherwise deformed.

The pressure sensor may include a deformable portion. The deformable portion may be elastomeric. At least the elastomeric portion may include an electrically conductive material. The electrically conductive material may only be electrically conductive when the pressure exerted on the housing exceeds a predetermined threshold. The pressure sensor may further include a projection that extends axially from the deformable portion in a direction away from the housing. The pressure sensor may further include at least one electrical trace configured to engage the deformable portion when the pressure exerted on the housing exceeds a predetermined threshold. When the pressure exerted on the housing is below the predetermined threshold, the deformable portion may be spaced apart from the at least one electrical trace. The monitor may also include at least one further electrical trace configured to engage the deformable portion when the pressure exerted on the housing is below the predetermined threshold.

The monitor may be configured to determine a blood glucose level of the patient. The monitor may further include an alarm configured to activate when the blood glucose level of the patient is determined to be below a predetermined blood glucose level. Activation of the alarm may be configured to be blocked when the pressure exerted on the housing exceeds a predetermined threshold.

According to another embodiment of the invention, a method of monitoring a blood glucose level of a patient in vivo using a monitor that includes a housing configured to adhere to a patient's skin, a sensor member configured to extend from the housing into the patient's skin, and a pressure sensor positionable between part of the housing and part of the patient's skin, includes the steps of: adhering the monitor to the patient's skin, with the sensor member extending from the housing into the patient's skin, and with the pressure sensor positioned between part of the housing and part of the patient's skin, and initiating the monitoring of the blood glucose level of the patient. When pressure is exerted on the housing towards the patient's skin, the pressure sensor is configured to measure a magnitude of the pressure exerted on the housing.

The monitor may further include an alarm configured to activate when the blood glucose level of the patient is determined to be below a predetermined blood glucose level. Activation of the alarm may be configured to be blocked when the pressure exerted on the housing exceeds a predetermined threshold.

According to embodiments of the invention, low signals caused by compression or other pressure at the sensor site can be filtered out and distinguished from low signals caused by low blood glucose levels of the patient. For example, if a low signal is detected concurrently with a high pressure notification, the monitor may be able to identify and filter out these low signals, and may for example, override or at least delay alarms associated with low blood glucose detections, until for example, the pressure is released from the sensor site. This would allow an opportunity, for example, for the patient to naturally change position during sleep, release the pressure from the sensor site, and allow the sensor and associated signal to recover organically without setting off an alarm or otherwise waking the patient. If, for example, the system determines that the pressure has been released from the sensor site, but the low signal does not recover, then a proper alarm or other notification can then be sent when such situation arises. In some embodiments, even if a high compression event is detected, the system may still activate the alarm after a set period of time if the low signal remains during that duration of time, for example, after 30 minutes or one hour. This would safeguard against low blood glucose levels that occur concurrently with a high compression event, so that the high compression event does not inadvertently mask or conceal the low blood glucose levels. When the patient is asleep, such a backup alarm may wake the patient to alert them to shift to a position where compression at the sensor site is released or alleviated if the patient remains in the high compression position for too long, but may not be set off if, for example, the patient shifts position while asleep and naturally releases the compression at the sensor site before the time threshold is reached. Using such an arrangement, unnecessary false alarms or other disturbances to the patient, particularly those that occur when the patient is asleep, can be reduced or eliminated.

### BRIEF DESCRIPTION OF THE DRAWINGS

Further features and advantages of the invention will become apparent from the description of embodiments by means of the accompanying drawings. In the drawings:

FIGS. 1A and 1B schematically show a human body with an analyte monitor including an analyte sensor according to embodiments of the invention, where the analyte monitor is attached at different positions on the body.

FIG. 2 shows a perspective view from above an exemplary analyte monitor including an analyte sensor according to embodiments of the invention.

FIG. 3 shows a perspective view from below the analyte monitor of FIG. 2.

FIG. 4 shows an enlarged perspective view of a portion of a sensor member of an analyte monitor according to an embodiment of the invention.

FIG. 5 shows a cutaway view of a portion of a patient's skin with a schematic depiction of a sensor member of an analyte monitor according to an embodiment of the invention implanted therein.

FIG. 6A shows a cross-sectional view of an additional pressure sensor incorporated into an analyte sensor according to an embodiment of the invention, while FIG. 6B shows an enlarged portion of the pressure sensor of FIG. 6A.

FIG. 7A shows a cross-sectional view of the pressure sensor of FIG. 6A when a pressure external to the analyte sensor is applied on the analyte sensor, and FIG. 7B shows an enlarged portion of the pressure sensor of FIG. 7A.

### DETAILED DESCRIPTION

In the following detailed description, only certain embodiments of the subject matter of the present disclosure are described, by way of illustration. As those skilled in the art would recognize, the subject matter of the present disclosure may be embodied in many different forms and should not be construed as being limited to the embodiments set forth herein.

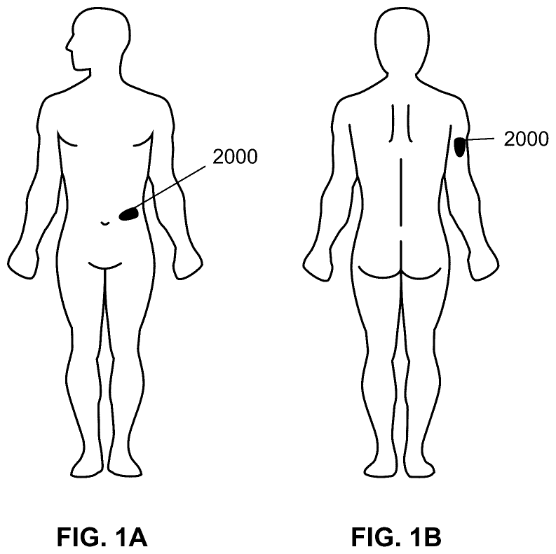

Monitors that include analyte sensors, such as glucose monitors, and in particular continuous glucose monitors, can be attached to a patient's body in different locations, in order to for example, improve glucose monitoring and/or a patient's comfort, since the continuous glucose monitors must remain adhered to the patient's skin, sometimes for a few days or more. FIG. 1A shows a first exemplary analyte monitor 2000 that is adhered to a patient's abdominal region, while FIG. 1B instead shows the exemplary analyte monitor 2000 adhered to a patient's arm. These are only meant to be example adhesion sites, and in other situations, this or a similar analyte monitor may instead be adhered or otherwise attached to other parts of the patient's body.

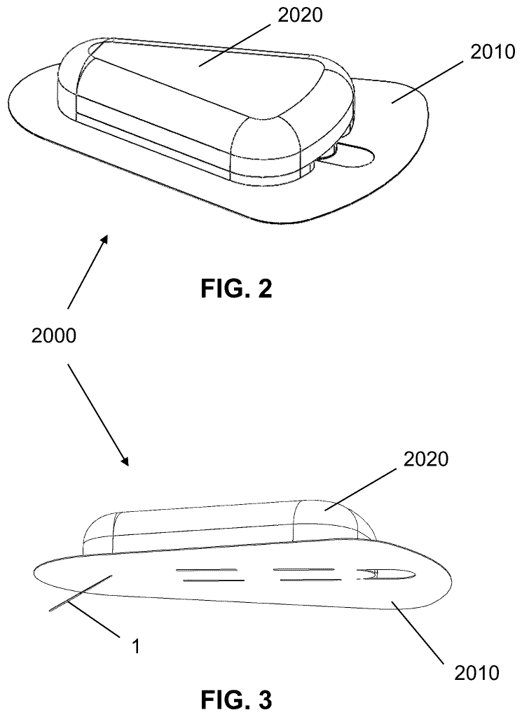

FIGS. 2 and 3 show different schematic views of an example analyte monitor 2000, which can be a continuous glucose monitor, according to an embodiment of the invention. The continuous glucose monitor 2000 may include a base or cradle 2010 that may have an adhesive layer for adhering to a patient's skin, a transmitter 2020 for transmitting data to and/or from a location away from the monitor, and a sensor member 1 which may include an integrated analyte sensing region such as a glucose sensor. It is to be understood that the example analyte monitor shown in FIGS. 2 and 3 are for illustrative and descriptive purposes only, and that analyte monitors with different structures and functionality can also be used in conjunction with the sensor assemblies described below, without departing from the spirit or scope of the invention.

The base 2010 will generally include an adhesive patch or other adhesive mechanism on its lower surface in order to facilitate attachment of the base 2010 to the surface of the patient's skin. In some embodiments of the invention, the sensor member 1 may be integrally formed with the base 2010 so as to be implanted under the patient's skin upon attachment of the base 2010 to the surface of the skin, while in other embodiments the base 2010 may be applied to the skin first, and the sensor member 1 may be advanced to a position where the sensor member 1 becomes attached to the base during or after implantation of part of the sensor member 1 under a patient's skin. For some embodiments, application of the base 2010 and/or the sensor member 1 may be facilitated with one or more applicators.

In some embodiments, after the base 2010 has been attached to a surface of the patient's skin and after at least part of the sensor member 1 has been advanced under the patient's skin, the transmitter 2020 which may be a separate part can be attached to the base. In some embodiments, the transmitter 2020 may include, for example, a power source such as a battery, while the base 2010 and/or the sensor member 1 may include additional electrical circuitry or contacts to complete a circuit, such that the monitor 2000 is powered up upon assembly of the transmitter 2020 to the base 2010. Other embodiments may include other arrangements, for example, where the battery is housed in the base 2010 instead of on the transmitter 2020, etc. Still other embodiments may include further different arrangements, for example, an integrally manufactured monitor where the base and transmitter are formed together in a single main body and which may not be separable from one another by the end user, where such embodiments may also include an integrated sensor member, or may include a sensor member that is implanted through the main body of the monitor after the main body has been adhered to the surface of the patient's skin.

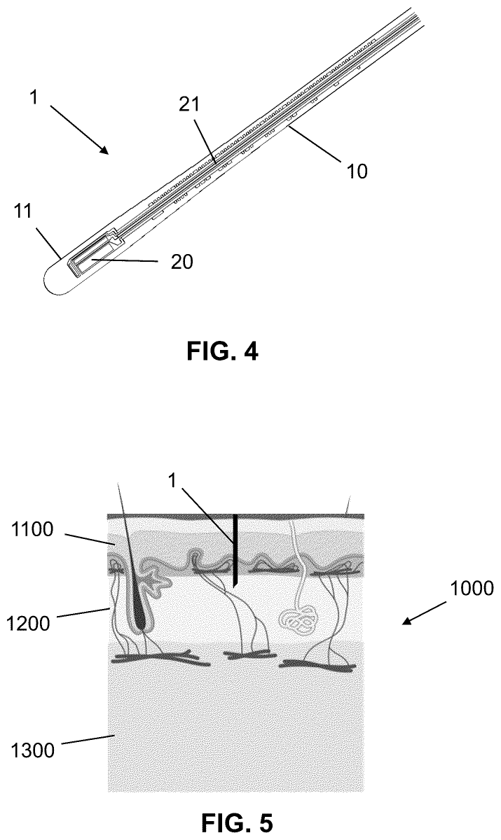

Construction of a sensor member 1 according to an embodiment of the invention will now be discussed in greater detail, with reference to FIG. 4. As seen in FIG. 4, a sensor member 1 may include an implantable portion that includes a main body 10 and a sensing region including one or more sensing electrodes 20. The sensing electrodes 20 may include one or more of a working electrode, a counter electrode, or a reference electrode, where other embodiments may include more or less electrodes, according to the particular application. The main body 10 may include a sensor tip or distal end region 11, where the sensing electrodes are typically located at or near the distal end region 11. In some embodiments, the distal end region 11 may be enlarged, for example, in a width direction compared to other portions of the main body. For example, in one embodiment, the distal end region 11 may be substantially teardrop-shaped or otherwise widened with a tapering profile. In other embodiments, the distal end region may include arms or teeth extending laterally outwardly, or may include other shapes or profiles that provide for a varying width. Enlarging the distal end region 11 can help anchor the sensing electrodes 20 at a particular desired position under the surface of the skin and facilitate improved positioning and retention of the sensor member 1 under the skin after implantation. The sensing electrodes 20 may further be electrically connected to leads 21 that extend along the main body 10 of the sensor and to an opposite proximal end of the sensor member 1, to facilitate electrical connectivity and communication of the sensing electrodes 20 with other electrical components of the monitor.

FIG. 5 shows a cutaway view of a portion of a patient's skin, with a schematic depiction of the sensor member 1 implanted therein, according to an embodiment of the invention. As shown in FIG. 5, the skin 1000 of a human includes various layers, such as an epidermis 1100 that is closest to the surface of the skin, an intermediate dermis 1200, and a deeper hypodermis 1300. Sensor members 1 according to embodiments of the invention may only extend into the shallow regions of the dermis 1200, with sensing regions in either the shallow regions of the dermis 1200, the epidermis 1100, or both, rather than extending more deeply into the hypodermis 1300, as is the case with many other monitors on the market. Positioning the sensing regions of the sensor member 1 in the shallow regions of the dermis 1200 or the epidermis 1100 may have many advantages. For example, the smaller size of the sensor member 1 according to embodiments of the invention, both in terms of length and thickness, may reduce trauma during insertion and/or discomfort during the life of the sensor/monitor. The reduction in size of the sensor member 1 compared to traditional sensor members, or more generally, the reduced depth to which the sensor member 1 extends under the skin, will also allow sensing in less fatty layers of the skin, consequently resulting in faster glucose transport, more accurate signal readings by the sensor member, and a shorter delay or time lag associated with being able to retrieve useable signals from the sensor member 1. Targeting the shallower regions of the dermis 1200 furthermore allows for some tolerance or leeway, where for example, if a sensor member 1 is implanted at a slightly more vertical angle than intended, the sensing regions of the sensor member 1 should still be positioned in the dermis 1200 rather than deeper in the hypodermis 1300, and still benefit from both reduced discomfort and less glucose transport delays due to fat in the tissue. Sensor members in other embodiments may be positioned shallower or deeper than as described, without departing from the spirit or scope of the invention.

However, with sensor members that are located more shallowly in the patient's skin, such positioning exposes the system to more external conditions or influences. Particularly, the monitor may be more sensitive to pressures applied against the skin at the site of the sensor member. As noted above, when the skin is compressed, the amount of interstitial fluid, particularly in the shallower portions of the skin, may be reduced, and accordingly, glucose level readings by the monitor may be affected via for example, the system reading lower blood glucose levels locally at the site of compression that may not be consistent with the actual higher blood glucose levels of the patient.

Therefore, in accordance with embodiments of the invention, an additional pressure sensor, such as a strain gauge or another type of force sensor, may be incorporated into or onto the analyte monitor. In particular, a pressure sensor may be added to a bottom or lower surface of the monitor housing, i.e., the surface of the monitor housing that is adhered to the surface of the patient's skin. This may be, for example, the lower surface of the base 2010 as shown in FIG. 3. When an adhesive layer is formed on the lower surface of the base 2010, the pressure sensor may be implemented at a portion of the base where the adhesive has been removed, for example, so that the pressure sensor directly engages the surface of the patient's skin. In other embodiments, the pressure sensor may instead be implemented above the adhesive layer, such that the adhesive layer remains between the pressure sensor and the patient's skin.

The pressure sensor, generally formed as a strain gauge or other force sensor, can detect when the sensor member is under a certain level of compression conditions, e.g., when a compression or other pressure force above a designated threshold is being applied against the sensor member under the patient's skin, or when the surrounding tissue is under a certain level of compression or pressure force above the designated threshold. In such situations, the system can identify that the sensor member is under increased pressure or compression, and that low signals detected by the monitor during this time are likely due to the increased compression conditions, rather than due to actual low blood glucose levels in the patient. This ability to filter out low signals due to high compression conditions, or in other words that signal artifacts may have been induced by increased pressure at the sensor site, can improve the quality of data being collected by the monitor, and thereby improve the experience of the end user.

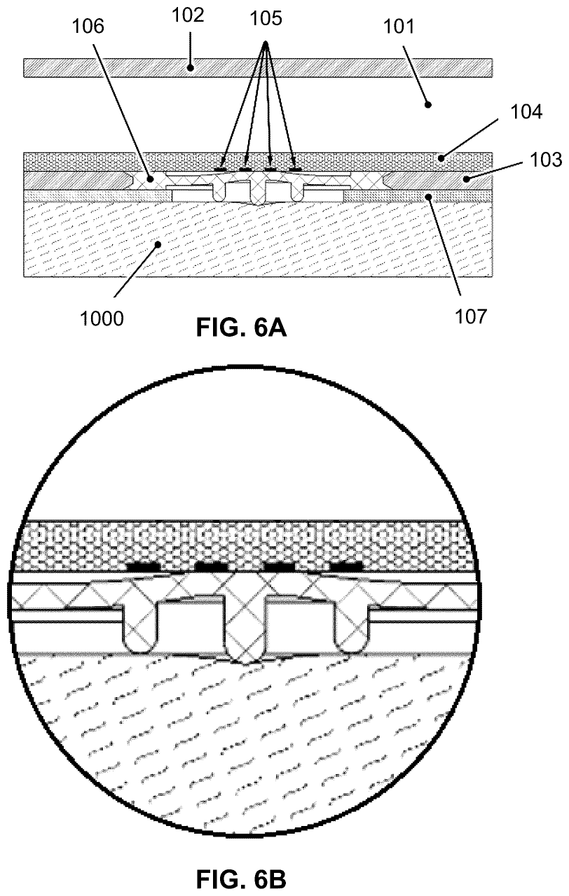

FIG. 6A shows a cross-sectional view of an example pressure sensor that is incorporated into an analyte sensor, according to an embodiment of the invention, while FIG. 6B shows an enlarged portion of the pressure sensor of FIG. 6A. As noted above, the pressure sensor may be incorporated into the base of the analyte sensor in some embodiments, but may be implemented elsewhere in other embodiments without departing from the spirit or scope of the invention. The analyte sensor 101 is shown schematically (i.e., without some internal components that are not essential to understanding the inventive concepts of the instant application), with the reference 101 pointing generally to the space of the housing of the analyte sensor, that is sandwiched between or defined by a top surface or shell 102 and a bottom surface or shell 103. In this arrangement, at least the bottom shell 103 may be formed as part of the base 2010 as shown in FIGS. 2 and 3. Held inside the analyte sensor 101 may be a printed circuit board or other form of electronics 104, which may include, among other components, leads connecting to a transmitter, a battery, and/or processing hardware.

In addition, the printed circuit board 104 may incorporate a number of conductive contacts or traces 105, where in the embodiment shown, four conductive traces 105 are shown in cross-section. However, more or less conductive traces may be included in other embodiments, for example, while four conductive traces 105 are shown in cross-section, there may be an array of additional conductive traces formed in the actual three-dimensional structure of the pressure sensor according to embodiments of the invention. In the embodiment shown, an opening is formed in the bottom shell 103, and a conductive diaphragm 106 is formed and provided in the opening. The conductive diaphragm 106 may include, for example, projections that extend away from the housing of the analyte sensor 101, and towards the surface of the patient's skin 1000 when the analyte sensor 101 is adhere thereto. In some embodiments, the conductive diaphragm 106 may be a separate part that is attached to the bottom shell 103 or another part of the analyte sensor 101, while in other embodiments, the conductive diaphragm 106 may instead be integrally formed with the bottom shell 103 or another part of the analyte sensor 101. In the former example, the conductive diaphragm 106 may include or be made of a separate elastomeric or other type of flexible material. In the latter example, part of the conductive diaphragm 106 may include or be made by thinning a local portion of the bottom shell 103, for example, so as to render the thinned portion flexible or more flexible, and thus more prone to flexion or deformation when the system is in compression and the stress applied against that part of the bottom shell 103 is increased. The analyte sensor 101 may further include, for example, an adhesive patch or layer 107 to facilitate attachment of the analyte sensor 101 to the patient's skin 1000. In the embodiment shown, a further opening is formed in the adhesive patch 107 to provide direct access by the conductive diaphragm 106 to the patient's skin 1000.

A general structure of the conductive diaphragm 106 of the pressure sensor and its interactions with both the conductive traces 105 and the patient's skin 1000 will now be described, with further reference to FIGS. 6A and 6B. As noted above, the conductive diaphragm 106 may include one or more projections that extend away from the rest of the analyte sensor 101 and that are configured to contact the patient's skin 1000. A length of the projections is such that when the housing of the analyte sensor 101 is pressed against the patient's skin 1000, the skin 1000 will push up against the projections and deform an opposite face of the conductive diaphragm 106 that faces the conductive traces 105, such that a greater portion of the conductive diaphragm 106 engages the region of the circuit board where the conductive traces 105 are located. In the embodiment shown, the projections of the conductive diaphragm 106 extend past an outer surface of the adhesive patch 107 in a neutral position, but in other embodiments, the projections may not extend that far, for example, in embodiments where there is no opening in the adhesive patch. In such latter embodiments, the projections may rest and/or exert a slight pressure against the conductive patch in the neutral position. Here, as seen most clearly in FIG. 6B, when the analyte sensor 101 is adhered to the patient's skin 1000, there may be a slight pressure caused by the patient's skin 1000 against the conductive diaphragm 106. Here, the opposite surface of the conductive diaphragm 106 may be in contact with a subset of the conductive traces 105, for example, two of the four conductive traces 105 as illustrated in FIG. 6B. The contact between the conductive parts 105/106 can be determined by the electronics of the analyte sensor 101 (or in some embodiments, by processing equipment located away from the analyte sensor 101), and in the uncompressed position shown in FIGS. 6A and 6B, the contact with a smaller subset of the conductive traces 105 by the conductive diaphragm 106 may indicate a proper application of the analyte sensor 101 to the skin 1000 of the patient. In other embodiments, there may be no contact between the conductive diaphragm 106 and the conductive traces 105 in the neutral position.

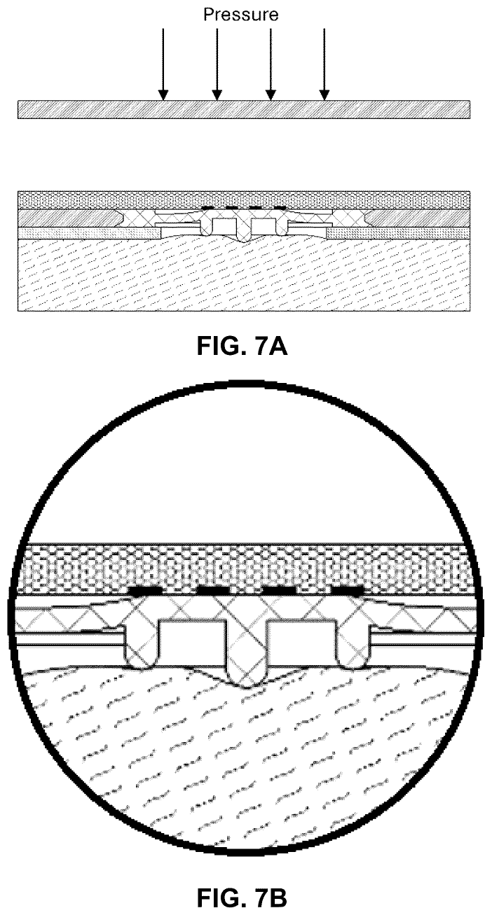

Referring then to FIGS. 7A and 7B, when a pressure is exerted on the analyte sensor 101, for example, against the top shell 102, the analyte sensor 101 may be pressed against the patient's skin 1000, causing compression of the underlying tissue in which the sensor member is located. Here, the skin 1000 will exert a counterforce against the projections of the conductive diaphragm 106 when under pressure, causing the conductive diaphragm 106 to be pushed upward (as illustrated), leading to increased contact with a greater number of conductive traces 105. Generally, a greater pressure will lead to contact with a greater number of conductive traces. When the pressure exceeds a certain threshold, for example, a threshold as defined by the manufacturer, the conductive diaphragm 106 will contact either all or at least a greater subset of the conductive traces 105. In some embodiments, the traces may be arranged in circular fashion, e.g., via concentric ring traces, such that an increased pressure will cause contact by more outer rings from among the concentric rings to engage the conductive diaphragm 106. When a defined number of conductive traces 105 is in contact with the conductive diaphragm 106, or in the example of circular traces, when the conductive diaphragm 106 engages one or more specific more outer positioned rings, the system may identify a high compression situation, where low recorded signals may be caused by the high compression situation. In some embodiments, there may further be a magnitude component or multiple pressure thresholds incorporated into the detection system, where for example, the number of conductive traces 105 contacted by the conductive diaphragm 106, or for example, the number of rings contacted by the conductive diaphragm 106, can be used to determine a magnitude of compression or different pressure levels above various different thresholds, which may further be helpful in compensating for high compression situations when monitoring the blood glucose readings from the analyte sensor.

Some embodiments may incorporate, for example, a conductive elastomer or other material that may only be conductive when exposed to high enough pressure. In such embodiments, the conductive elastomer may be pre-loaded against the skin such that, in a neutral situation, the conductive elastomer experiences a pressure that is less (e.g., slightly less) than the threshold at which the elastomer becomes conductive. Thereafter, when the elastomer is deformed a sufficient amount, generally as selected or designated by the manufacturer, the elastomer will become conductive and will conduct electricity across it, thereby indicating to the system that it is under a certain level of compression or load. The blood glucose signals and/or alarms can then be adjusted accordingly. In another embodiment, the conductive elastomer may have a variable resistance depending on the amount of deformation, so as to be more conductive the more the elastomer is deformed, e.g., as pressure applied to the elastomer increases.

Other embodiments of pressure sensors can also be implemented without departing from the spirit or scope of the invention. For example, in some embodiments, particularly those with locally thinned housing portions, a strain gauge may be attached to the portion of the housing that has been thinned and rendered more flexible, and may be configured to detect compression conditions at the flexible portion of the housing. The readings from such a strain gauge can then be utilized to determine high compression situations, similarly to the previously described embodiments. Still other pressure sensors can also be implemented in similar fashion.

As described above, according to embodiments of the invention, a pressure sensor may be incorporated into the analyte sensor, and can be used to detect high compression or high pressure situations, where an external load applied onto the analyte sensor may affect the tissue surrounding the sensor member and cause inaccurate low signals to arise. While the pressure sensors described herein have been incorporated into the housing of the analyte sensor, other arrangements may also be possible, for example, incorporating a pressure sensor onto the sensor member itself, such that compression of the tissue surrounding the sensor member can be detected directly by the pressure sensor located on the sensor member. Other arrangements and variations are also possible without departing from the spirit or scope of the invention. In addition, various further modifications can also be incorporated while retaining the inventive concepts of the present application.

In addition to the embodiments that have already been described above, it is also possible to combine embodiments, e.g., different features from the various described embodiments, to provide even more different variations of analyte monitors, without departing from the spirit or scope of the invention. In addition, the inventions should not be limited to the structures and/or shapes described in the embodiments above.

While the subject matter of the present disclosure has been described in connection with certain embodiments, it is to be understood that the subject matter of the present disclosure is not limited to the disclosed embodiments, but, on the contrary, the present disclosure is intended to cover various modifications and equivalent arrangements included within the spirit and scope of the appended claims, and equivalents thereof.

What is claimed is:

1. A monitor for determining analyte concentrations in vivo, comprising:
a housing configured to adhere to a patient's skin;
a sensor member configured to extend from the housing into the patient's skin; and
a pressure sensor positionable between part of the housing and part of the patient's skin;
wherein when pressure is exerted on the housing towards the patient's skin, the pressure sensor is configured to measure a magnitude of the pressure exerted on the housing.

2. The monitor of claim 1, wherein the pressure sensor is formed on a surface of the housing configured to face towards the patient's skin.

3. The monitor of claim 2, wherein the pressure sensor is integrally formed on the surface of the housing configured to face towards the patient's skin.

4. The monitor of claim 1, further comprising an adhesive layer configured to be positioned between the housing and the patient's skin, to facilitate adhesion of the monitor to the patient's skin.

5. The monitor of claim 4, wherein an opening is formed in the adhesive layer, and wherein the pressure sensor extends from the housing through the opening, such that the pressure sensor is configured to directly contact the patient's skin.

6. The monitor of claim 5, wherein the adhesive layer has an outer surface that faces away from the housing and that is configured to engage the patient's skin, and wherein at least part of the pressure sensor is configured to extend axially past the outer surface of the adhesive layer, such that when the adhesive layer contacts the patient's skin, the pressure sensor is at least partially actuated or otherwise deformed.

7. The monitor of claim 1, wherein the pressure sensor comprises a deformable portion.

8. The monitor of claim 7, wherein the deformable portion is elastomeric.

9. The monitor of claim 8, wherein at least the elastomeric portion comprises an electrically conductive material.

10. The monitor of claim 9, wherein the electrically conductive material is only electrically conductive when the pressure exerted on the housing exceeds a predetermined threshold.

11. The monitor of claim 7, wherein the pressure sensor further comprises a projection that extends axially from the deformable portion in a direction away from the housing.

12. The monitor of claim 7, wherein the pressure sensor further comprises at least one electrical trace configured to engage the deformable portion when the pressure exerted on the housing exceeds a predetermined threshold.

13. The monitor of claim 12, wherein when the pressure exerted on the housing is below the predetermined threshold, the deformable portion is spaced apart from the at least one electrical trace.

14. The monitor of claim 12, further comprising at least one further electrical trace configured to engage the deformable portion when the pressure exerted on the housing is below the predetermined threshold.

15. The monitor of claim 1, wherein the monitor is configured to determine a blood glucose level of the patient.

16. The monitor of claim 15, further comprising an alarm configured to activate when the blood glucose level of the patient is determined to be below a predetermined blood glucose level.

17. The monitor of claim 16, wherein activation of the alarm is configured to be blocked when the pressure exerted on the housing exceeds a predetermined threshold.

18. A method of monitoring a blood glucose level of a patient in vivo using a monitor comprising a housing configured to adhere to a patient's skin, a sensor member configured to extend from the housing into the patient's skin, and a pressure sensor positionable between part of the housing and part of the patient's skin, the method comprising:
adhering the monitor to the patient's skin, with the sensor member extending from the housing into the patient's skin, and with the pressure sensor positioned between part of the housing and part of the patient's skin; and
initiating the monitoring of the blood glucose level of the patient;
wherein when pressure is exerted on the housing towards the patient's skin, the pressure sensor is configured to measure a magnitude of the pressure exerted on the housing.

19. The method of claim 18, wherein the monitor further comprises an alarm configured to activate when the blood glucose level of the patient is determined to be below a predetermined blood glucose level.

20. The method of claim 19, wherein activation of the alarm is configured to be blocked when the pressure exerted on the housing exceeds a predetermined threshold.
